(19) United States
(12) Patent Application Publication
(10) Pub. No.: US 2004/0170688 A1
(43) Pub. Date: Sep. 2, 2004

(54) Enteric formulation of fluoxetin

(76) Inventors: Abhijit Mukund Deshmukh, Hyderabad (IN); Ujwal Damu Kolhe, Hyderabad (IN); Vipin Tatyasaheb Dhanorkar, Hyderabad (IN); Mailatur Sivaraman Mohan, Hyderabad (IN)

Correspondence Address: LADAS & PARRY, 26 WEST 61ST STREET, NEW YORK, NY, 10023, US

(21) Appl. No.: 10/486,157

(22) PCT Filed: Apr. 19, 2002
(86) PCT No.: PCT/IB02/01268

(30) Foreign Application Priority Data
Aug. 6, 2001 (IN) 647/MAS/2001

(51) Int. Cl. A61K 9/24

(52) U.S. Cl. 424/471

(57) ABSTRACT

An industrially advantageous enteric formulation of Fluoxetin without the use of hydroxypropylmethylcellulose acetate succinate and sucrose is covered by this invention. The present invention also covers said enteric formulations of Fluoxetin in the form of tablets or capsules with an optional separating layer. When in the form of capsules, the separating layer is capsule shell itself thus reducing processing step of said enteric formulations. The formulation of the present invention along with Fluoxetin or its pharmaceutically accepted salts, solvates, enantiomers or mixtures thereof including racemic mixture is also contemplated to be within the scope of present invention.

### FIELD OF THE INVENTION

The present invention belongs to the field of Pharmaceutical Sciences and provides an industrially advantageous improved formulation of Fluoxetin or a pharmaceutically acceptable salt, solvate, enantiomer or mixtures thereof including raceric mixture, which is designed for once a week dosing. The present invention teaches that active ingredient is in the form of pluralities of particles as spherical, cylindrical or elliptical units, pellets, minitablets, tablets or capsules, which can be enteric coated with enteric polymers with an optional smoothening layer. The enteric polymer can be applied in a manner, which does not require any neutralization or reduction of free acidic groups.

### BACKGROUND OF THE INVENTION

Fluoxetin, N-Methyl-3-phenyl-3-[(α,α,α-trifluorotolyl) oxyl] propylamine, is an antidepressant drug, which is disclosed in U.S. Pat. Nos. 4,314,081, 4,626,549 and 5,847,217. The teaching for (S) and (R) enantiomeric forms of Fluoxetin is found in U.S. Pat. No. 5,889,186 and 5,708,035 respectively. Method and formulation for treating depression using optically pure Fluoxetin is disclosed in U.S. Pat. No. 5,104,899. Published literature supports the fact that Fluoxetin and its active metabolite, NorFluoxetin show very long elimination half-life and eliminates slowly from the body even after discontinuation of the dosing. Because of the long half-life of Fluoxetin, there has not been any perceived need to actually prepare a Fluoxetin formulation providing a longer payout. While these higher doses of Fluoxetin have been shown to be efficacious, there can be associated side effects, such as nausea, presumably due to local irritation or the increased plasma levels shortly after dosing. Therefore, it has now been appreciated that a formulation having higher doses of Fluoxetin (e.g. 60 mg to 120 mg) which blunts the initial release of Fluoxetin will have clinical advantages, i.e. not only will such formulations provide convenient and effective once per week dosing, but will have an advantage of less side effects.

Enteric pharmaceutical formulation of Fluoxetin with a dose of 90 mg is disclosed in U.S. Pat. No. 5,985,322. Anderson et al. in U.S. Pat. No. 5,985,322 discloses the enteric Fluoxetin pellets wherein, the Fluoxetin with one or more pharmaceutically acceptable excipients is coated over the inert nonpareil seeds made of sucrose and starch. The drug coated core is then optionally coated with a separating layer comprising of non reducing sugar, sucrose along with one or more pharmaceutically acceptable excipients and coated with hydroxypropylmethylcellulose acetate succinate as enteric coating polymer along with one or more pharmaceutically acceptable excipients and finally coated with finishing layer hydroxypropylmethylcellulose and talc.

U.S. Pat. No. 5,910,319 teaches that certain difficulties arose in preparing conventional enteric formulations of Fluoxetin. In particular, Fluoxetin was found to react with many enteric coatings to form a slowly—or even insoluble coating. Therefore it is important that the separating layer should be used to prevent such an interaction of enteric polymer and Fluoxetin. The separating layer is also used to provide smooth surface for the enteric coat, to improve the acid resistance of the pellets. It has been noted that the use of sucrose in separating layer has surprisingly improved the acid resistance of the pellets and successfully prevented the direct contact of core pellets with enteric polymer. One of the further objectives of the smoothening layer described in U.S. Pat. No. 5,910,319 is to improve the coverage of enteric layer and to avoid thin spots in it caused by bumps and irregularities on the core.

The U.S. Pat. No. 5,985,322 discloses the use of hydroxypropylmethylcellulose acetate succinate as most preferred enteric polymer in view of 4% to 28% of succinoyl groups, which are the only free carboxylic groups in the compound. It is disclosed that the enteric polymer must be the one having only small number of carboxylic acid groups per unit weight or repeating units of the polymers so as to decrease the chances of reaction of Fluoxetin and enteric polymer to form a slowly dissolving or even insoluble coating.

Specific teaching of U.S. Pat. No. 5,910,319 is directed towards the problem that Fluoxetin can precipitate in needle-like crystals during processing, which can be quite large. Coating cores with Fluoxetin in the large needle-like form can be difficult, and it is advisable to mill or otherwise reduce the particle size of the Fluoxetin to less than about 50 μm before using it in the present product and process.

It should be noted that the teachings of U.S. Pat. No. 5,910,319 and 5,985,322 are associated with substantial problems like selection of enteric polymers having only small number of carboxylic acid groups per unit weight of repeating unit of polymer. The hydroxypropyl methyl cellulose acetate succinate, which is suggested by the said patent, has the amount of succinoyl groups from 4% to 28% which are the only free carboxylic acid groups present in the compound. The other problems are the incorporation of separating layer, which increases the processing steps and reducing the particles size of Fluoxetin to less than 50 μm before using.

The present invention does not require the reduction of particle size of Fluoxetin to less than 50 μm before using. Moreover Fluoxetin has a particle size wherein 90% particles are of size less than 229 microns, 50% particles are of size less than 90 microns and 10% particles are of size less than 23 microns.

The smoothening coat is an optional feature of the invention. Particularly when the formulation is in the form of capsule the gelatin capsule shell itself act as a separating layer and avoids the extra processing steps in the enteric formulation and enables the formulator to incorporate enteric polymer with substantially high number or free carboxylic acid groups.

Similarly the present invention also avoids the usual long processing time required for the coating of drug layer over nonpareil seeds thus saving the processing time and production cost. Likewise, the usual problems of drug loss, which occurs during the coating of drug on the inert non-pareil seeds as described in U.S. Pat. No. 5,985,322 and 5,910,319 are avoided.

### SUMMARY OF THE INVENTION

It is an objective of the present invention to produce an improved enteric formulation of Fluoxetin without the use of hydroxypropyl methyl cellulose acetate succinate and sucrose.

The formulation of the present invention comprises;

(a) a core comprising Fluoxetin or a pharmaceutically accepted salt, solvate, enantiomers or mixtures thereof including racemic mixture, in an amount of 90 mg base equivalent of Fluoxetin;

(b) an optional smoothening layer;

(c) an enteric coating layer comprising an at least one enteric coating polymers selected from the group consisting of Eudragit L100-55, Eudragit L 100, Eudragit S 100, hydroxypropyl methyl cellulose pthalate, cellulose acetate pthalate, polyvinyl acetate pthalate; an at least one plisticisers selected from the group consisting of triethyl citrate, polyethylene glycol, diethyl phthalate or dibutyl phthalate; an at least one lubricant or glidants selected from the group consisting of talc, magnesium stearate, kaolin or colloidal silicon dioxide.

(d) an optional finishing layer.

### DETAILED DESCRIPTION OF THE INVENTION

The present invention is designed in the form of enteric formulations manufactured in such a way that the product passes unchanged through the stomach of the patient, and dissolves and releases the active ingredient quickly when it leaves the stomach and enters the small intestine. The enteric formulations of the present invention are in the form of tablet or capsule wherein, the active ingredient, Fluoxetin or a pharmaceutically accepted salts, solvates, enantiomers or mixtures thereof including racemic mixture, is in the inner part of the tablet, minitablets, pellet or pluralities of particles as spherical, elliptical or cylindrical units, enclosed with a film or envelope called as the “enteric coating”, which is insoluble in acid environments, such as the stomach, but is soluble in near-neutral environments such as the small intestine. When the enteric formulation is in the form of capsule, the capsules are banded/sealed with gelatin solution followed by enteric coating, such that the capsule shell itself acts as separating layer to avoid the possible reaction of the active ingredient and the enteric polymer. Such a presence of natural separating coat avoids the excess manufacturing steps required in earlier inventions. Further it has been noted that the capsules enteric coated in the said manner are found to be therapeutically equivalent to the commercially available product Prozac® Weekly 90 mg capsule and have acceptable stability as per ICH guidelines.

When used in this specification, the term “active ingredient” refers to Fluoxetin or a pharmaceutically accepted salts, solvates, enantiomers or mixtures thereof including racemic mixture. The invention contemplates the enteric formulations comprising Fluoxetin preferably as hydrochloride salts, however as will be appreciated by those skilled in the art, other salt form or free base form could be used to obtain the same beneficial feature of the invention. Moreover, solvates of Fluoxetin or its salts or free bases, salts, and/or solvates of the individual isomers of Fluoxetin, namely (R)-Fluoxetin and (S)-Fluoxetin, are contemplated by this invention Throughout this description, unless specified otherwise, the term “Fluoxetin” contemplates all such forms, although Fluoxetin hydrochloride is clearly the most preferred embodiment of this invention. It should be noted that there is no difference in the word Fluoxetin or Fluoxetine and can be used interchangeably in this specification without altering the scope and meaning of the invention.

The present invention utilizes Fluoxetin in the range of particle size wherein 90% particles are of size less than 229 microns, 50% particles are of size less than 90 microns and 10% particles are of size less than 23 microns.

The present enteric coated formulations can be prepared by coating the enteric polymer having substantially high free carboxylic acid groups and does not require to limit the free carboxylic acid group in the range of from 4% to 28%.

According to one of the embodiments of the present invention the active ingredient is in the form of pluralities of particles as spherical, elliptical or cylindrical units. The delivery system in the form of plurality of single units offers many clinical advantages. Each of the single units act as a separate entity therefore the chances of dose dumping or unpredictable transit across the gastrointestinal tract due to variable gastric or intestinal residence time is overcome by using plurality of single units.

Preferably, the present invention describes the manufacturing of core of active material in the form of mini-tablets or pluralities of particles as spherical elliptical or cylindrical units, either by compressing the active agent with one or more of the pharmaceutically acceptable excipients on tablet compression machine or by extrusion-spheronization technique. The present invention describes an improved enteric formulation containing Fluoxetin or pharmaceutically accepted salts or solvates thereof, in the dosage range of 60-120 mg, preferably 90-120 mg and most preferably 90 mg base equivalent of Fluoxetin. The enteric formulations according to the present invention are designed for the treatment of various depressive disorders known in the art with a dosing frequency of once every seven days.

The formulation of the present invention is in the form of capsules or tablets comprising pluralities of particles as spherical, elliptical or cylindrical units or in the form of mini-tablets. When the formulation is in the form of pluralities of particles the size of such particles ranges from 0.5 mm to 3.0 mm. When the formulation is in the form of minitablets the size of such minitablets is in the range of from 0.5 mm to 6 mm preferably 0.5 mm to 4 mm. When the formulation is in the form of tablets the size of such tablets is in the range of 6 mm to 16 mm preferably 8 mm to 14 mm more preferably 8 mm to 11 mm. These pluralities of particles as spherical elliptical or cylindrical units are filled in the hard gelatin capsules of size ranging from 3 to 000. The hard gelatin capsules are then sealed with the gelatin solution in water in the concentration of 5-50% w/w at temperature ranging from 37° C. to 70° C. using hard gelatin capsule band sealing machine known to the pharmaceutical Industry. Alternately the sealing can be done using aqueous or nonaqueous solution of any of the polymers selected from hydroxypropylnethylcellulose, hydroxypropyl cellulose or hydroxyethylcellulose.

The word sealing and banding are used interchangeably in this description, which means applying a “band” of cohesive or polymeric materials as aqueous or non-aqueous solution to fuse the cap and the body of the capsule. Sealing of hard gelatin capsules with a band of gelatin or other cellulosic materials is known to the pharmaceutical industry since long and is very common technique employed in order to make the capsules tamperproof. However, the object of applying such a sealing or banding of the capsules according to the present invention is to prevent the migration of enteric solution to the interior of the capsule thus avoiding the contact of active ingredient with the enteric coating. Further in the present invention the capsules are sealed to fuse the cap and body of the gelatin shell to provide uniform surface for subsequent coatings.

When the active ingredient is in the form of tablets it is preferred that smoothening layer/coat is applied. When applied, said smoothening, coat/layer is composed of cohesive or polymeric material with finely divided solid excipients, which constitute fillers. The polymeric or cohesive materials can be selected from any of the polymeric materials selected from hydroxypropylmethylcellulose, polyvinylpyrrolidone, hydroxypropylcellulose, polyethylene glycol, sodium alginate, Eudragit RD 100, combination of N-vinyl pyrollidone and vinyl acetate, combination of microcrystalline cellulose and carragenan etc. Hydroxypropylmethylcellulose and polyethylene glycol are the preferred material for smoothening layer as per present description. The fillers used are those commonly used in pharmaceutical industries like finely powdered talc, silicon dioxide etc. The preferred aspect of the present invention is to avoid the use of sucrose in said smoothening layer, the use of which may be detrimental to the patients having history of hyperglycemia.

When enteric formulation is in the form of capsule the smoothening layer may be applied to facilitate more even enteric coat. The capsule shell itself acts as separating barrier (separating coat), which prevents the interaction of acidic enteric polymer with the active ingredient in the core. As used in this description the term “separating barrier” means a capsule shell with or without a band.

The formulation of the present invention avoids the need of separating layer between the enteric layer and the core containing active ingredient (Fluoxetin it pharmaceutically accepted salt, solvates, enantiomers and mixtures thereof including racemic mixture) thus reduces the extra processing steps needed to manufacture such formulations containing separating layer. The enteric layer is composed of a water-insoluble polymer together with a plasticizer and one or more pharmaceutically accepted excipients. The polymers used for enteric coating as per the present invention are selected from the group consisting of Eudragit L100-55, Eudragit L 100, Eudragit S 100, hydroxypropyl methyl cellulose pthalate, cellulose acetate pthalate, polyvinyl acetate pthalate etc. The preferred polymer is Eudragit L100-55.

The coating process can be as follows. The Eudragit L 100-55 is dissolved in solvent such as isopropyl alcohol and triethyl citrate and magnesium stearate are added to it. The resulting solution is sprayed on the tablets or capsules using coating pan. Alternatively aqueous dispersion of Eudragit L 100-55 (Spray dried Eudragit L30D-55 which can be reconstituted for aqueous formulations) can also be used for coating. Eudragit L 100-55, Triethyl citrate or magnesium stearate can be replaced by the functionally equivalent ingredients as described in this specification.

The various components and layers of the enteric formulation of the present invention are discussed individually as follows.

Pluralities of Particles:

The pluralities of particles as spherical, elliptical or cylindrical units are prepared by using wet granulation with or without use of binders like N-vinyl pyrollidone, hydroxypropyl methyl cellulose (5 cps-100 cps), hydroxypropylcellulose, pregelatinized starch, starch paste, combination of N-vinyl pyrollidone and vinyl acetate and gelatin in the concentration range of 2% to 20%.

The said formulation also contains one or more of the pharmaceutically accepted diluents like sorbitol, mannitol, microcrystalline cellulose, dicalcium phosphate or combination thereof. The formulation of the present invention also contains surfactants like sodium lauryl sulphate, poloxamer 407, Tween 20/40/60/80, Span 20/40/60/80, Cremophor RH 40 or combination thereof.

The said formulation also contains one or more of the pharmaceutically accepted disintegrants selected from crosscarmellose sodium, crosspovidone, sodium carboxymethylcellulose, sodium starch glycolate or such like.

In the present formulation when water is used as granulating solution the percentage of water with respect to weight of powder mass ranges from 10% to 50% w/w. The moisture content of the wet mass ranges from 10% to 50%. The pluralities of particles are manufactured by extrusion of wet mass of Fluoxetin and one or more pharmaceutically acceptable excipients followed by spheronization.

Composition of Core Pellets

| Ingredients | Quantity taken |
|---|---|
| Fluoxetin hydrochloride USP/NF equivalent to | 10-80% w/w |
| Fluoxetin base 90 mg | |
| Mannitol USP | 30-80% w/w |
| Microcrystalline cellulose (Avicel PH 101) NF | 0-70% w/w |
| Hydroxypropylmethyl cellulose NF, 5 cps | 2-10% w/w |
| Crosspovidone NF | 1-10% w/w |
| Sodium lauryl sulphate or Poloxamer 407 NF | 0.1-2% w/w |
| Purified water | Qs |

All the ingredients except hydroxypropyl methylcellulose (5 cps) were weighed and blended together for 15-30 minutes. The blend was then sifted through 40 # screen. The sifted powder mass was then granulated with hydroxypropylmethylcellulose solution in water. The wet mass was then passed through extruder. The extrudes were then spheronized in spheronizer at speed ranging from 200 rpm to 1500 rpm for the period of 2 minutes to 15 minutes. The pellets were then dried for sufficient period of time till the loss on drying of pellets was not more than 1.5% w/w.

### EXAMPLE 1

Core Pellet: (Plurality of Particles as Spherical, Elliptical or Cylindrical Units)

| | Quantity |
|---|---|
| | taken in |
| Ingredients | mg |
| Fluoxetin hydrochloride USP/NF equivalent to | 101 |
| Fluoxetin base 90 mg | |
| Mannitol USP | 345 |
| Microcrystalline cellulose (Avicel PH 101) NF | 32 |
| Hydroxypropylmethyl cellulose NF, 5 cps | 16 |
| Crosspovidone NF | 8.5 |
| Sodium lauryl sulphate or Poloxamer 407 NF | 2.5 |
| Purified water | qs |

Optional Smoothening Layer:

| | Quantity |
|---|---|
| | taken |
| Ingredients | in mg |
| Hydroxypropyl methyl cellulose 5 cps | 9 |
| Polyethylene glycol 400 | 1.35 |
| Talc | 0.45 |
| Purified water | qs |

Enteric Coat:

| | Quantity |
|---|---|
| | taken |
| Ingredients | in mg |
| EUDRAGIT ® 100-55 | 17.1 |
| Triethyl citrate | 1.71 |
| Magnesium stearate | 1 |
| Isopropyl alcohol | qs |

### EXAMPLE 2

Core Pellet: (Plurality of Particles as Spherical, Elliptical or Cylindrical Units)

| | Quantity |
|---|---|
| | taken in |
| Ingredients | mg |
| Fluoxetin hydrochloride USP/NF equivalent to | 101 |
| Fluoxetin base 90 mg | |
| Mannitol USP | 345 |
| Microcrystalline cellulose (Avicel PH 101) NF | 32 |
| Hydroxypropylmethyl cellulose NF, 5 cps | 16 |
| Crosspovidone NF | 8.5 |
| Sodium lauryl sulphate or Poloxamer 407 NF | 2.5 |
| Purified water | qs |

Optional Smoothening Layer:

| | Quantity |
|---|---|
| | taken |
| Ingredients | in mg |
| Hydroxypropyl cellulose 5 cps | 9 |
| Polyethylene glycol 400 | 1.35 |
| Colloidal silicon dioxide | 0.45 |
| Purified water | qs |

Enteric Coat:

| | Quantity |
|---|---|
| | taken |
| Ingredients | in mg |
| Hydroxypropyl methyl cellulose | 17.1 |
| pthalate | |
| Dibutyl phthalate | 1.71 |
| Magnesium stearate | 1 |
| Isopropyl alcohol | qs |
| Acetone | qs |

### EXAMPLE 3

Core Pellet: (Plurality of Particles as Spherical, Elliptical or Cylindrical Units)

| | Quantity |
|---|---|
| Ingredients | taken in mg |
| Fluoxetin hydrochloride USP/NF equivalent to | 101 |
| Fluoxetin base 90 mg | |
| Mannitol USP | 345 |
| Hydroxypropylmethyl cellulose NF, 5 cps | 16 |
| Crosspovidone NF | 8.5 |
| Sodium lauryl sulphate or Poloxamer 407 NF | 2.5 |
| Purified water | 106.6 |

Optional Smoothening Layer:

| | Quantity |
|---|---|
| Ingredients | taken in mg |
| Hydroxypropyl methyl cellulose 5 cps | 9 |
| Polyethylene glycol 400 | 1.35 |
| Talc | 0.45 |
| Purified water | qs |

Enteric Coat:

| | Quantity |
|---|---|
| | taken |
| Ingredients | in mg |
| Cellulose acetate pthalate | 17.1 |
| Triethyl citrate | 1.71 |
| Talc | 1 |
| Isopropyl alcohol | qs |
| Acetone | qs |

Mali-Tablets:

The mini-tablets are prepared either by using wet granulation or direct compression or dry granulation method with or without use of binders like N-vinyl pyrrolidone, hydroxypropylmethyl cellulose (5 cps to 100 cps) hydroxypropylcellulose, pregelatinized starch, starch paste, combination of N-vinyl pyrrolidone and vinyl acetate and gelatin in the concentration range of 2% to 20%. The Said formulation also contains one or more of the pharmaceutically accepted excipients like mannitol, sorbitol, microcrystalline cellulose, dicalcium phosphate or combination thereof as diluents. Magnesium stearate, stearic acid, lubritab, talc and silicon dioxide are used as lubricants and glidants. The said pharmaceutical excipients also contain disintegrants like hydroxy propyl cellulose, crosspovidone, sodium starch glycolate, crosscarmellose sodium or combination thereof. The said formulation also contains surfactants like sodium lauryl sulphate, poloxamer 407,Tween 0/40/60/80, Span 20/40/60/80, Cremophor RH 40 or combination thereof.

Preparation of Mini-Tablets or Tablets

| Ingredients | Quantity taken |
|---|---|
| Fluoxetin hydrochloride USP/NF equivalent to | 10-80% w/w |
| Fluoxetin base 90 mg | |
| Microcrystalline cellulose NF | 10-90% w/w |
| (Avicel PH 102/112/200) | |
| Polyvinylpyrollidone NF (PVP K 30/K 90)/ | 2-10% w/w |
| Plasdone S-630 | |
| Crosspovidone NF | 2-10% w/w |
| Magnesium stearate NF | 0.1-3% w/w |
| Talc NF | 0.1-3% w/w |
| Colloidal silicon dioxide NF | 0.1-5% w/w |
| Sodium lauryl sulphate or Poloxamer 407 NF | 0.1-5% |

All the ingredients except magnesium stearate and talc were weighed accurately and sifted through 40 # screen. The sifted materials were, then blended for 5-60 minutes in a suitable blender. Magnesium stearate and talc were weighed and sifted through 40 # screen and added to other ingredients and blended for 5-20 minutes before compressing into mini-tablets. Alternatively all the excipients except magnesium stearate, talc and polyvinyl pyrollidone were weighed accurately and sifted through 40 # screen and were granulated while polyvinylpyrrolidone solution in water. The wet mass was then optionally milled and dried or dried directly till the loss on drying was 0.3% to 5% w/w. The dried granules were again milled and mixed with magnesium stearate. Talc is weighed and sifted separately. The lubricated granules were mixed for 5 minutes to 20 minutes and compressed into tablets or mini-tablets.

The core of the present invention can be coated with film coating polymers like N-vinyl pyrrolidone (PVP K-30/K-90), polyethylene glycol, hydroxypropylmethylcellulose, hydroxypropylcellulose, hydroxyethylcellulose, sodium alginate, EUDRAGIT® RD 100, combination of N-vinyl pyrrolidone and vinyl acetate along with one or more pharmaceutically acceptable excipients like plasticisers, glidants, anti adherent agents to improve the process of capsule filling

### EXAMPLE 4

Fluoxetin Mini-Tablets Formulation:

| | Quantity |
|---|---|
| | taken |
| Ingredients | In mg |
| Fluoxetin hydrochloride USP/NF equivalent to | 101 |
| Fluoxetin base 90 mg | |
| Microcrystalline cellulose NF | 294 |
| (Avicel PH 102/112/200) | |
| Polyvinylpyrollidone NF (PVP K30/K90)/ | 27 |
| Plasdone S-630 | |
| Crosspovidone NF | 18 |
| Magnesium stearate NF | 2 |
| Talc NF | 2 |
| Colloidal silicon dioxide NF | 6 |
| Sodium lauryl sulphate or Poloxamer 407 NF | 2 |

Optional Smoothening Layer:

| | Quantity |
|---|---|
| | taken |
| Ingredients | in mg |
| Hydroxypropyl methyl cellulose 5 cps | 9 |
| Polyethylene glycol 400 | 1.35 |
| Talc NF | 0.45 |
| Purified water | qs |

Enteric Coat:

| | Quantity |
|---|---|
| | taken |
| Ingredients | in mg |
| Eudragit L 100-55 | 17.1 |
| Triethyl citrate | 1.71 |
| Magnesium stearate | 1 |
| Isopropyl alcohol | qs |

### EXAMPLE 5

### Fluoxetin Tablet Formulation

| | Quantity |
|---|---|
| | taken |
| Ingredients | In mg |
| Fluoxetin hydrochloride USP/NF equivalent to | 101 |
| Fluoxetin base 90 mg | |
| Microcrystalline cellulose NF | 294 |
| (Avicel PH 102/112/200) | |
| Polyvinylpyrollidone NF (PVP K30/K90)/ | 27 |
| Plasdone S-630 | |
| Crosspovidone NF | 18 |
| Magnesium stearate NF | 2 |
| Talc NF | 2 |
| Colloidal silicon dioxide NF | 6 |
| Sodium lauryl sulphate or Poloxamer 407 NF | 2 |

Smoothening Layer:

| | Quantity |
|---|---|
| | taken |
| Ingredients | in mg |
| Plasdone S-630 | 12.59 |
| Triethyl citrate | 1.89 |
| Talc | 1.26 |
| Purified water | qs |

Enteric Coat:

| | Quantity |
|---|---|
| | taken |
| Ingredients | in mg |
| Eudragit L 100-55 | 27.2 |
| Triethyl citrate | 2.72 |
| Magnesium stearate | 0.25 |
| Isopropyl alcohol | Qs |

### EXAMPLE 6

### Fluoxetin Tablets Formulation

| | Quantity |
|---|---|
| | taken |
| Ingredients | In mg |
| Fluoxetin hydrochloride USP/NF equivalent to | 101 |
| Fluoxetin base 90 mg | |
| Microcrystalline cellulose NF | 294 |
| (Avicel PH 102/112/200) | |
| Polyvinylpyrollidone NF (PVP K30/K90)/ | 27 |
| Plasdone S-630 | |
| Crosspovidone NF | 18 |
| Magnesium stearate NF | 2 |
| Talc NF | 2 |
| Colloidal silicon dioxide NF | 6 |
| Sodium lauryl sulphate or Poloxamer 407 NF | 2 |

Smoothening Layer:

| | Quantity |
|---|---|
| | taken |
| Ingredients | in mg |
| Hydroxypropyl methyl cellulose 5 cps | 12.59 |
| Polyethylene glycol 400 | 1.89 |
| Talc | 1.26 |
| Purified water | qs |

Enteric Coat:

| | Quantity |
|---|---|
| | taken |
| Ingredients | in mg |
| Cellulose acetate pthalate | 27.2 |
| Dibutyl phthalate | 2.72 |
| Colloidal silicon dioxide | 0.25 |
| Isopropyl alcohol | Qs |
| Acetone | Qs |

Hard Gelatin Capsule Sealing:

The core in the form of pluralities of particles as spherical, elliptical or cylindrical units are filled in hard gelatin capsules of size ranging from 3 to 000. The hard gelatin capsules are then sealed with gelatin solution in water in the concentration range of 5-50% w/w at temperature ranging from 37° C. to 70° C. using hard gelatin capsule band sealing machine. The sealing of capsules is done to fuse the cap and body of capsules to provide uniform surface for the further functional coating and also to prevent the possible ingress of solvent during coating.

Optional Smoothening Coat/Layer:

The smoothening coat when applied is optional with an object of providing a smooth surface for the enteric coating. The optional smoothening coat can be applied using one or more of the agents like N-vinyl pyrrolidone (PVP K-30/K-90), polyethylene glycol, hydroxypropylmethylcellulose, hydroxypropylcellulose, hydroxyethylcellulose, sodium alginate, Eudmgit RD100, combination of N-vinyl pyrrolidone and vinyl acetate (Plasdone S-630), Opadry AMB along with one or more pharmaceutically acceptable excipients like plasticisers selected from triethyl citrate, polyethylene glycol, diethyl phthalate, dibutyl phthalate, glidants, antiadherents like talc, magnesium stearate, kaolin, colloidal silicon dioxide, which are commonly known to those skilled in the art. These agents are applied in the range of 0.5 to 9 mg/cm² surface area corresponding to 0.5 to 7% polymer with respect to weight of core.

Enteric/Site Specific Coating:

In the present invention the agents which inhibits the release of drug in stomach and releases the same once the pH in the body reaches 5.0 to 7.5 are sodium alginate, cellulose acetate, copolymers derived from methacrylic acid/ethyl acrylate, anionic methacrylic acid and methacrylic acid esters, cellulose acetate phthalate, polyvinyl acetate phthalate and hydroxypropylmethylcellulose phthalate or combination thereof.

These agents are used in the range of 1 mg/cm² to 20 mg/cm² and are more preferably from 2 mg/cm² to 12 mg/cm² of surface area corresponding to about 2% to 20% of the enteric polymer with respect to the weight of core. The coating solution/suspension also contains excipients like plasticisers selected from triethyl citrate, polyethylene glycol, diethyl phthalate, dibutyl phthalate, glidants, antiadherents like talc, magnesium stearate, kaolin or colloidal silicon dioxide and such like.

Optional Finishing Coat:

The finishing coat can be optionally applied with an object to improve the elegance of the product.

The agents which constitute the finishing coat includes various grades and colours of commercial product Opadry of M/s Colorcon which consists of hydroxy propyl methylcellulose along with one or more pharmaceutically acceptable excipients.

Stability Profile of the Enteric Formulations:

The assay and related substances show that the Fluoxetin 90 mg capsules and tablets are substantially stable over the storage period of 3 months and 6 months respectively at the storage conditions of 40° C. and 75% RH (relative humidity).

Assay and Related Substances for Fluoxein 90 mg Capsules at Storage Condition of 40° C. and 75% RH.

| | Initial | After 3 months |
|---|---|---|
| Assay | 101.6% | 103.9% |
| Related substances | 0.03% | 0.07% |

Assay and Related Substances Fluoxetin 90 mg Tablets at Storage Condition of 40° C. and 75% RH.

| | Initial | After 6 months |
|---|---|---|
| Assay | 98.20% | 103.3% |
| Related substances | 0.13% | 0.15% |

The percent acid release of drug at pH 6.8 as well as gastric resistance is not affected adversely even after exposing the tablet to 40° C. and 75% RH for 6 months and capsules for 40° C. and 75% RH for 3 months.

Gastric Resistance and Dissolution Profile of Fluoxetin 90 mg Capsules at Storage Condition of 40° C. and 75% RH.

| | Initial | After 3 months |
|---|---|---|
| % acid release in 0.1 N | 0.0% | 0.0% |
| HCL for 2 hours using | | |
| USP apparatus I at pH | | |
| 6.8 at 100 RPM. | | |
| % dissolution at pH | | |
| 6.8 using USP | | |
| apparatus I at 100 | | |
| RPM | | |
| 30 min | 80% | 67% |
| 60 min | 93% | 87% |
| 90 min | 95% | 89% |

Gastric Resistance and Dissolution Profile of Fluoxetin 90 mg Tablet at Storage Condition of 40° C. and 75% RH.

| | Initial | After 6 months |
|---|---|---|
| % acid release in 0.1 N | 0.0% | 0.0% |
| HCL for 2 hours using | | |
| USP apparatus I at pH | | |
| 6.8 at 100 RPM. | | |
| % dissolution at pH | | |
| 6.8 using USP | | |
| apparatus I at 100 | | |
| RPM | | |
| 30 min | 68% | 67% |
| 60 min | 82% | 79% |
| 90 min | 91% | 88% |

From the above data it is evident that gelatin shell acts as natural separating barrier/coat to prevent the interaction between Fluoxetin and enteric coating polymers (or polymers used for site specific coating as described in this specification) containing even substantially high percentages of free carboxylic acid groups.

The enteric Fluoxetin formulations of present invention was found to be substantially stable and therapeutically equivalent to the commercially available formulation Prozac® Weekly 90 mg capsules. The examples given above are for the purpose of illustration only and not to be construed as limitations thereon. Many variation of the present invention mentioned in the detailed description are obvious to those skilled in the art and are contemplated to be within the scope of the present invention.

We claim:

1. An enteric Fluoxetin formulation comprising:
(a) a core comprising Fluoxetin or a pharmaceutically accepted salt, solvate, enantiomers or mixtures thereof including racemic mixture, in an amount of 90 mg base equivalent of Fluoxetin,
(a) an optional smoothening layer,
(a) an enteric coating layer comprising an at least one enteric coating polymers selected from the group consisting of Eudragit L100-55, Eudragit L 100, Eudragit S 100, hydroxypropyl methylcellulose pthalate, cellulose acetate pthalate, polyvinyl acetate pthalate; an at least one plasticisers selected from the group consisting of triethyl citrate, polyethylene glycol, diethyl pthalate or dibutyl pthalate; an at least one lubricant or glidants selected from the group consisting of talc, magnesium stearate, kaolin or colloidal silicon dioxide, and
(a) an optional finishing layer.

2. The formulation according to claim 1 wherein, the core comprises pluralities of particles as spherical, elliptical or cylindrical units from 0.5 mm to 3.00 mm.

3. The formulation according to claim 1 wherein, the core comprises mini-tablets comprising from 0.5 nm to 6 nm.

4. The formulation as in any of claims 1 to 3 wherein the optional smoothening layer comprises;
(a) an at least one polymer selected from the group consisting of N-vinyl pyrollidone, polyethylene glycol, hydroxypropylmethyl cellulose, hydroxypropyl cellulose, hydroxyethyl cellulose, sodium alginate, Eudragit RD100 or combination of N-vinyl pyrollidone and vinyl acetate,
(b) an at least one filler selected from the group consisting of talc, magnesium stearate, kaolin or colloidal silicon dioxide, and
(c) an at least one plasticizer selected from the group consisting of triethyl citrate, polyethylene glycol, diethyl phthalate or dibutyl phthalate.

5. The formulation as in any of claims 1 to 4 in the form of hard gelatin capsule comprising a band, the band comprising aqueous or non-aqueous solution of a sealing polymers, said sealing polymers are selected from the group consisting of gelatin, hydroxypropylmethyl cellulose or hydroxypropylcellulose in an amount of 5% to 50% w/w.

6. The formulation as in any of the claims 1 to 5 comprising;

| Ingredients | Quantity taken | |
|---|---|---|
| Fluoxetin hydrochloride USP/NF equivalent to | 10-80% | w/w |
| Fluoxetin base 90 mg | | |
| Mannitol USP | 30-80% | w/w |
| Microcrystalline cellulose (Avicel PH 101) NF | 0-70% | w/w |
| Hydroxypropylmethyl cellulose NF, 5 cps | 2-15% | w/w |
| Crosspovidone NF | 1-10% | w/w |
| Sodium lauryl sulphate or Poloxamer 407 NF | 0.1-5% | w/w |
| Purified water | 10-50% | w/w |

7. An enteric Fluoxetin formulation comprising:
(a) a core comprising Fluoxetin or a pharmaceutically accepted salt, solvate, enantiomers or mixtures thereof including racemic mixture, in an amount of 90 mg base equivalent of Fluoxetin,
(b) a smoothening layer,
(c) an enteric coating layer comprising an at least one enteric coating polymers selected from the group consisting of Eudragit L100-55, Budragit L 100, Eudragit S 100, hydroxypropyl methylcellulose pthalate, cellulose acetate pthalate, polyvinyl acetate pthalate; an at least one plasticisers selected from the group consisting of triethyl citrate, polyethylene glycol, diethyl pthalate or dibutyl pthalate; an at least one lubricant or glidants selected from the group consisting of talc, magnesium stearate, kaolin or colloidal silicon dioxide, and
(d) an optional finishing layer.

8. The formulation of claim 7 wherein the smoothening layer comprises;
(a) an at least one cohesive or polymeric material selected from the group consisting of hydroxypropylmethylcellulose, hydroxypropylcellulose, polyethylene glycol, sodium alginate, Eudragit RD 100, polyvinylpyrrolidone or combination of N-vinyl pyrollidone and vinyl acetate, combination of microcrystalline cellulose and carragenan, and,
(b) an at least one pharmaceutically accepted filler selected from the group consisting of talc, magnesium stearate, kaolin or colloidal silicon dioxide

9. The formulation as in any of claims 1 or 7 comprising Fluoxetin hydrochloride.

10. The formulation as in any of claims 7 or 8 comprising;

| | Quantity | |
|---|---|---|
| Ingredients | taken | |
| Fluoxetin hydrochloride USP/NF equivalent to Fluoxetin | 10-80% | w/w |
| base 90 mg | | |
| Microcrystalline cellulose NF | 10-90% | w/w |
| (Avicel PH 102/112/200) | | |
| Polyvinylpyrollidone NF (PVP K-30/K-90)/Plasdone | 2-15% | w/w |
| S-630 | | |
| Crosspovidone NF | 2-10% | w/w |
| Magnesium stearate NF | 0.1-3% | w/w |
| Talc NF | 0.1-3% | w/w |
| Colloidal silicon dioxide NF | 0.1-5% | w/w |
| Sodium lauryl sulphate or Poloxamer 407 NF | 0.1-5% | |

11. In an enteric capsule formulation comprising Fluoxetin or a pharmaceutically accepted salt, solvate, enantiomers or mixtures thereof including racemic mixture, in an amount of 90 mg base equivalent of Fluoxetin; the improvement comprises applying enteric layer to the capsule shell thus avoiding the need of applying separating layer between the enteric layer and the core containing drug to prevent the possible reaction between the drug and the acidic enteric polymer of the enteric coat.

12. In an enteric tablet formulation comprising Fluoxetin or a pharmaceutically accepted salt, solvate, enantiomers or mixtures thereof including racemic mixture, in an amount of 90 mg base equivalent of Fluoxetin; the improvement comprises avoiding sucrose in the smoothening layer.
